(19) United States
(12) Patent Application Publication
(10) Pub. No.: US 2013/0089929 A1
(43) Pub. Date: Apr. 11, 2013

(54) MICRODEVICE FOR FUSING CELLS

(71) Applicants: Sang-Woo Joo, Gyeongsan-si (KR); Shizhi Qian, Gyeongsan-si (KR); Ning Hu, Gyeongsan-si (KR)

(72) Inventors: Sang-Woo Joo, Gyeongsan-si (KR); Shizhi Qian, Gyeongsan-si (KR); Ning Hu, Gyeongsan-si (KR)

(21) Appl. No.: 13/282,833

(22) Filed: Oct. 27, 2011

(30) Foreign Application Priority Data
Oct. 6, 2011 (KR) 10-2011-0101881

(51) Int. Cl. C12M 1/42; C12N 15/02

(52) U.S. Cl. 435/450; 435/285.2

(57) ABSTRACT

A microdevice for fusing cells, the microdevice including: a substrate; a first electrode array including a plurality of first electrodes, and disposed on the substrate; a microwell array including a plurality of microwells formed respectively at locations corresponding to the plurality of first electrodes, and disposed on the first electrode array; a second electrode disposed above the plurality of microwells, and including a microchannel having a predetermined height; inlet and outlet holes mutually spaced apart from the microchannel; and a power supply unit applying voltage to the plurality of first electrodes and the second electrode. Accordingly, a cell trapped in the microwell and a cell disposed on the microwell are aligned in a line between the first and second electrodes, and thus the two cells having different traits are smoothly fused in a one-to-one manner when an electric shock is applied to the two cells.

### CROSS-REFERENCE TO RELATED PATENT APPLICATION

This application claims the benefit of Korean Patent Application No. 10-2011-0101881, filed on Oct. 6, 2011, in the Korean Intellectual Property Office, the disclosure of which is incorporated herein in its entirety by reference.

### BACKGROUND OF THE INVENTION

1. Field of the Invention

The present invention relates to a microdevice for fusing cells for electrofusion, which manufactures a desired fused cell at high efficiency.

2. Description of the Related Art

Cell fusion is a method of preparing a hybrid cell by artificially fusing two cells in different types. The cell fusion may be performed by using chemicals or an electric pulse. Here, combining two cells in different types by porating a cell membrane via an electric pulse is referred to as electrofusion.

There are mainly four continuous phases in the electrofusion: dielectrophoresis-based cell alignment, reversible electroporation, reconstruction of cytomembrane, and karyon fusion. Generally, the dielectrophoresis-based cell alignment needs a sinusoidal alternating current (AC) electric field (intensity: 100 to 300 V/cm) to exert a positive dielectrophoretic (DEP) force on the cells. In addition, a high-strength DC electric pulse signal series is required in the reversible electroporation (intensity: 1 to 10 kV/cm, pulse width: 10 to 50 μs).

Plate electrodes are usually used in a conventional cell electrofusion device. In general, a distance between two plate electrodes is equal to or above 1 cm, and as a result, an expensive generator is required to obtain high-strength electric pluses. Moreover, an electric field generated between the plate electrodes is uniform, and thus probabilities of occurrence of reversible electroporation and electrofusion of aligned cells are equal. Thus, a probability of occurrence of unwanted multi-cell electrofusion in the conventional cell electrofusion device is relatively high.

In order to increase pairing precision, fusion efficiency, multi-function integration, and a degree of automation, a micro electromechanical system (MEMS) and microfluidic technology have been used to develop microchips for electrofusion. Microstructures in these microchips have a similar scale as cells (5 to 50 μm), and thus useful in more precise cell manipulation. Also, owing to a short distance between two microelectrodes, a high electric field required for cell fusion may be generated even with a low voltage, and thus difficulties of power supply and high manufacturing costs may be reduced.

However, in a conventional microfluidic device, an average cell fusion efficiency is about 40%, which is higher than a general chemical fusing method (use polyethylene glycol (PEG), less than 5%) and a conventional electrofusion method (less than or equal to 12%), but a probability of forming desired cell-cell twins is only from 42 to 68%. Accordingly, fusion efficiency of total cells is about 40%×42-68%, i.e., 16 to 30%. In other words, when a cell A and a cell B are to be fused, undesired hybrid products, such as AA, ABB, AABB, MB, and BB, may be excessively obtained instead of AB.

Accordingly, a new microfluidic chip for fusing desired cells at higher efficiency is required to be developed.

### SUMMARY OF THE INVENTION

The present invention provides a microdevice for fusing cells, wherein cells to be fused are effectively fused in a one-to-one manner.

According to an aspect of the present invention, there is provided a microdevice for fusing cells, the microdevice including: a substrate; a first electrode array including a plurality of first electrodes, and disposed on the substrate; a microwell array including a plurality of microwells formed respectively at locations corresponding to the plurality of first electrodes, and disposed on the first electrode array; a second electrode disposed above the plurality of microwells, and including a microchannel having a predetermined height; inlet and outlet holes mutually spaced apart from the microchannel; and a power supply unit applying voltage to the plurality of first electrodes and the second electrode.

According to another aspect of the present invention, there is provided a method of fusing cells, the method including: providing the microdevice; injecting first cells through an inlet hole and flowing the first cells through a microchannel; applying an alternating current (AC) voltage between a first electrode and a second electrode such that the injected first cells are trapped in each microwell according to a dielectrophoresis ; injecting second cells through the inlet hole and flowing the second cells through the microchannel; performing electroporation by applying a direct current (DC) voltage between the first electrode and the second electrode when the second cells flow in and are disposed on the microwell in which the first cells are aligned; applying a AC voltage between the first electrode and the second electrode such that the electroporated first and second cells are fused by being adjacently disposed to each other according to a dielectrophoresis; and obtaining the fused first and second cells through an outlet hole.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view of a microdevice for fusing cells, according to an embodiment of the present invention;

FIG. 2 is an exploded perspective view of the microdevice, according to an embodiment of the present invention;

FIG. 3 is a perspective view of a lower structure according to an embodiment of the present invention;

FIG. 4 is a perspective view of a substrate according to an embodiment of the present invention;

FIG. 5 is a perspective view of a first electrode array according to an embodiment of the present invention;

FIG. 6 is a perspective view of a microwell array according to an embodiment of the present invention;

FIG. 7 is a perspective view of a structure of a second electrode according to an embodiment of the present invention;

FIG. 8 is a cross-sectional view of the microdevice according to an embodiment of the present invention; and

FIG. 9 is a perspective view of a structure of an upper cover according to an embodiment of the present invention.

### DETAILED DESCRIPTION OF THE INVENTION

Hereinafter, embodiments of the present invention will be described in detail. The invention may, however, be embodied in many different forms and should not be construed as being limited to the embodiments set forth herein; rather, these embodiments are provided so that this disclosure will be thorough and complete, and will fully convey the concept of the invention to those skilled in the art.

The present invention will be described more fully with reference to the accompanying drawings.

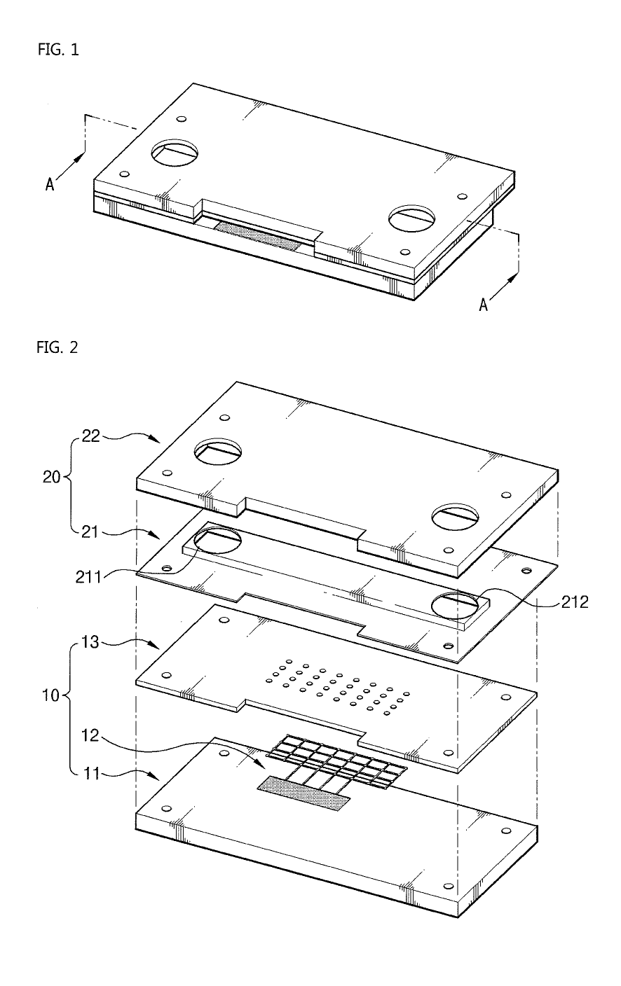

FIG. 1 is a perspective view of a microdevice for fusing cells, according to an embodiment of the present invention, and FIG. 2 is an exploded perspective view of the microdevice. For convenience of illustration, a power supply unit connected between first and second electrodes is not shown, but the power supply unit would have been obvious to one of ordinary skill in the art.

An embodiment of the present invention provide a microdevice for fusing cells, the microdevice including: a substrate 11; a first electrode array 12 including a plurality of first electrodes 121, and disposed on the substrate; a microwell array 13 including a plurality of microwells 131 formed respectively at locations corresponding to the plurality of first electrodes, and disposed on the first electrode array; a second electrode 21 disposed above the plurality of microwells, and including a microchannel having a predetermined height; inlet 211 and outlet 212 holes mutually spaced apart from the microchannel; and a power supply unit applying voltage to the plurality of first electrodes 121 and the second electrode 21.

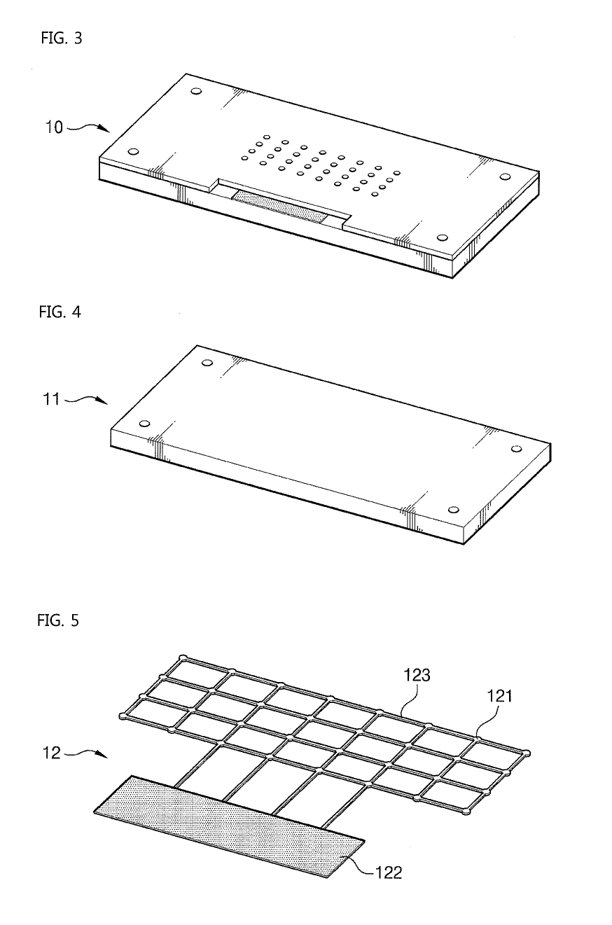

According to the current embodiment of the present invention, a lower structure 10 of the microdevice is formed by sequentially stacking a substrate 11, a first electrode array 12 including a plurlaity of first electrodes 121, and a microwell array 13 including a plurality of microwells 131 respectively formed at locations corresponding to the first electrodes 121. An example of the lower structure 10 is shown in FIG. 3, and FIGS. 4 through 6 are respectively perspective views of the substrate 11, the first electrode array 12, and the microwell array 13. A structure of each element will now be described in detail with reference to FIGS. 2 through 6.

The substrate 11 is disposed at the lowest bottom of the microdevice, is formed of an insulator, and performs an operation as a supporter of the first electrode array 12. A material for forming the substrate 11 is not limited as long as it is an insulating material, and in detail, the material may be silicon, silicon oxide, or glass quartz. A thickness of the substrate 11 is not limited as long as it performs the operation as a supporter, and may be equal to or above 400 μm.

The first electrode array 12 includes the first electrodes 121, and in detail, the first electrodes 121 may be arranged in a lattice shape and electrically connected to each other. Alternatively, the first electrode array 12 may include the first electrodes 121 electrically connected to each other, and a holding pad 122 electrically connected to the first electrodes 121 and receiving a predetermined voltage from the power supply unit. FIG. 5 illustrates such a first electrode array 12 including the first electrodes 121 electrically connected to each other and the holding pad 122 electrically connected to the first electrodes 121 and receiving a predetermined voltage from the power supply unit.

Referring to FIG. 5, the first electrodes 121 are arranged in a lattice shape of M×N, and are mutually connected by a conductive wire 123. The first electrode 121 is disposed below the microwell 131, and may be formed according to a size of the microwell 131. In detail, the first electrode 121 may have a diameter from 4 to 8 μm. A thickness of the conductive wire 123 is not limited as long as it has conductive power, and may be from 0.2 to 2 μm. A length of the conductive wire 123 corresponds to an interval between the first electrodes 121, and when the first electrodes 121 are arranged in a lattice shape, the interval between the first electrodes 121 may be from 20 to 40 μm. A size of the holding pad 122 is not limited as long as it can receive a voltage from the power supply unit and transmit the voltage to the first electrodes 121, and may be from 4 to 8 mm×from 2 to 6 mm.

The first electrode array 12 may be formed of a material that is biocompatible, dysoxidative, noncorrosive, and electric conductive. Examples of such a material include gold, platinum, and titanium, and are not limited thereto.

The first electrode array 12 may be formed by coating a thin film formed of the material for forming the first electrode array 12 on the substrate 11, and then etching the thin film in a pattern of the first electrode array 12 by using a standard lithography method.

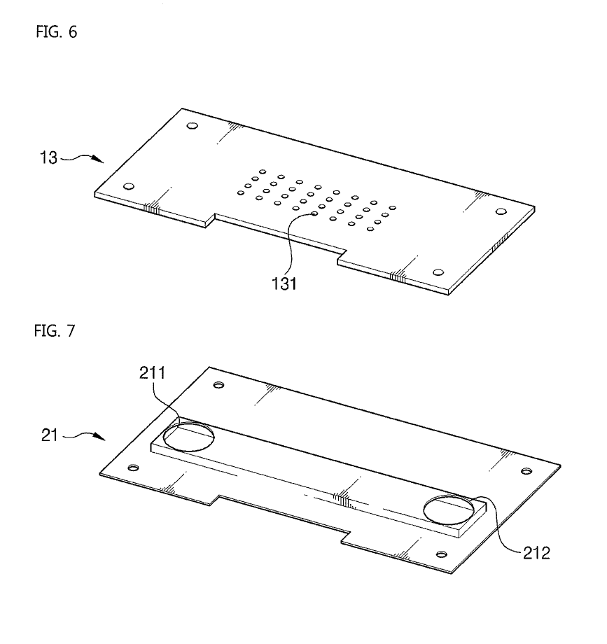

When the first electrodes 121 are arranged in the lattice shape of M×N, the microwell array 13 may also include the microwells 131 respectively formed at locations corresponding to the first electrodes 121 and arranged in the lattice shape of M×N, as shown in FIG. 6. Accordingly, the first electrodes 121 are respectively disposed below the microwells 131.

According to an embodiment of the present invention, the microwell 131 may have a cylindrical shape or a polygonal column shape. A depth and width of the microwell 131 corresponds to those of a single cell. Accordingly, geometric parameters, such as an aspect ratio, diameter, and depth, of the microwell 131 may be adjusted according to a size of a cell to be trapped, and in detail, the microwell 131 may have a depth and width of 8 to 30 μm.

The microwell array 13 may be formed of a material that is biocompatible, dysoxidative, noncorrosive, and electric resistive. In detail, Durimide 7510 may be used as the material, but the material is not limited thereto. Alternatively, a photosensitive material may be used.

According to an embodiment of the present invention, a second electrode 21 disposed above the microwells 131 and including a microchannel having a predetermined height forms an upper structure 20 of the microdevice.

The upper structure 20 may further include an upper cover 22 on the second electrode 21.

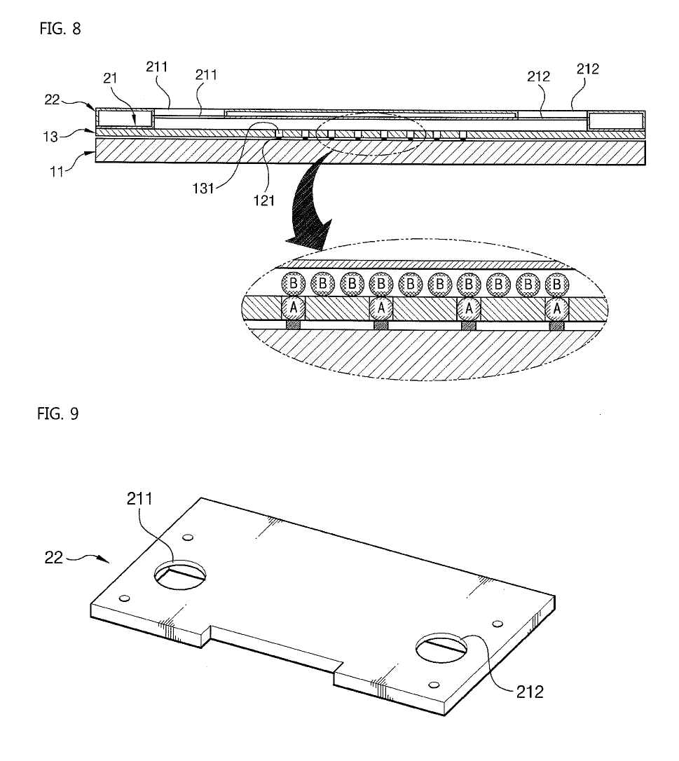

FIG. 7 is a perspective view of a structure of the second electrode 21 according to an embodiment of the present invention, FIG. 8 is a cross-sectional view of the microdevice according to an embodiment of the present invention, and FIG. 9 is a perspective view of a structure of the upper cover 22 according to an embodiment of the present invention.

The second electrode 21 is disposed on the microwells 131, and includes the microchannel having a predetermined height. The microchannel includes an inlet hole 211 and an outlet hole 212, which are spaced apart from each other, and the height of the microchannel may correspond to a diameter of a single cell.

A width and length of the microchannel may include all areas of the microwells 131.

Cells introduced through the inlet hole 211 flow along the microchannel, and at this time, a single second cell is disposed on the microwell 131 where a first cell is aligned. Accordingly, the height of the microchannel corresponds to that of a single cell so that the cells flow in a line. FIG. 8 is a cross-sectional view of such a microdevice where the cells flow in a line.

The second electrode 21 may be a metal film having a structure including the microchannel, and may be formed of a material that is biocompatible, dysoxidative, noncorrosive, and electric conductive. Examples of such a material include gold, platinum, and titanium, and but not limited thereto. A thickness of the metal film may be from 0.2 to 2 μm for excellent electric conductivity, but is not limited thereto.

The upper cover 22 may be further disposed on the second electrode 21.

According to an embodiment of the present invention, the upper cover 22 performs operations corresponding to the substrate 11, is disposed at the topmost portion of the microdevice, and supports the microdevice as an insulator. A material for forming the upper cover 22 is not limited as long as it is an insulating material, and in detail, the material may be silicon, silicon oxide, or glass quartz. A thickness of the upper cover 22 is not limited as long as it performs the operation as a supporter, and may be equal to or above 400 μm.

The inlet hole 211 and the outlet hole 212 respectively for injecting and discharging samples of the first and second cells are formed through the second electrode 21 and the upper cover 22. Diameters of the inlet and outlet holes 211 and 212 are not limited, but may be from 3 to 5 mm. The inlet and outlet holes 211 and 212 may be fabricated by using laser beam.

At least one hole may be formed through the substrate 11, the microwell array 13, the second electrode 21, and the upper cover 22 at the same corresponding locations, and at least one bolt or the like may be inserted through the at least one hole, thereby preparing the microdevice in which each of structures are stacked on each other. The at least one hole may be formed at four corners of each structure, but is not limited thereto.

An embodiment of the present invention provides a method of fusing cells, the method including: providing the microdevice; injecting first cells through an inlet hole and flowing the first cells through a microchannel; applying an alternating current (AC) voltage between a first electrode and a second electrode such that the injected first cells are trapped in each microwell according to a dielectrophoresis and gravity; injecting second cells through the inlet hole and flowing the second cells through the microchannel; performing electroporation by applying a direct current (DC) voltage between the first electrode and the second electrode when the second cells flow in and are disposed on the microwell in which the first cells are aligned; applying a quasi-damping AC voltage between the first electrode and the second electrode such that the electroporated first and second cells are fused by being adjacently disposed to each other according to a dielectrophoresis; and obtaining the fused first and second cells through an outlet hole.

The first cells injected through the inlet hole flow through the microchannel, and are trapped in each microwell according to gravity or dielectrophoresis. Here, in order to form dielectrophoresis, the AC voltage (amplitude: 2-20V, frequency: 0.2-3 MHz)is applied between the first and second electrodes, thereby forming an ununiform electric field between the first and second electrodes.

Since each microwell has a depth and width corresponding to those of a single cell, a process of removing remaining first cells that are not trapped in each microwell may be performed later. Such a process may be performed by using a syringe pump or electrophoresis between the inlet hole and the outlet hole formed via a DC voltage, but is not limited thereto.

After the first cell is trapped in the microwell, the second cells may be introduced through the inlet hole and flow through the microchannel. Since the microchannel has a height corresponding to a diameter of a single cell, the second cells injected into the microchannel are arranged in a single layer on the microwell array filled with the first cells. Accordingly, one second cell contact each of the microwells filled with the first cells, in a vertical direction. Dielectrophoresis may be induced by applying the AC voltage (amplitude: 2-20V, frequency: 0.2-3 MHz) so as to accelerate fusing of the first and second cells, and then a process of removing the second cells that are not paired from the microchannel according to electrophoresis may be additionally performed later.

Then, the DC voltage (amplitude: 6-50V, duration: 10-500 μs, interval of two pulses: 0.1-10 s, pulses: 1-100) is applied between the first and second electrodes such that the first and second cells vertically contacting as described above are reversibly electroporated. Then, the quasi-damping AC voltage (amplitude: 1-10 V, frequency: 0.2-3 MHz, attenuation rate: −0-90%/min) is applied between the first and second electrodes, thereby fusing the first and second cells by maintaining the connection of the first and second cells according to dielectrophoresis.

Next, a solution having a high dielectric constant is injected through the inlet hole, and the AC voltage (amplitude: 1-10V, frequency: 0.2-3 MHz) is applied between the first and second electrodes, thereby obtaining the fused first and second cells through the outlet hole according to negative dielectrophoresis. A PBS buffer solution may be used as the solution having a high dielectric constant, and the fused first and second cells may be obtained by using a syringe pump or electrophoresis, but are not limited thereto.

According to the present invention, a cell trapped in a microwell and a cell disposed on the microwell can be aligned in a line between first and second electrodes, and thus when an electric field is applied, the cells having different traits can be smoothly fused in an one-to-one manner.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A microdevice for fusing cells, the microdevice comprising:
a substrate;
a first electrode array comprising a plurality of first electrodes, and disposed on the substrate;
a microwell array comprising a plurality of microwells formed respectively at locations corresponding to the plurality of first electrodes, and disposed on the first electrode array;
a second electrode disposed above the plurality of microwells, and comprising a microchannel having a predetermined height;
inlet and outlet holes mutually spaced apart from the microchannel; and
a power supply unit applying voltage to the plurality of first electrodes and the second electrode.

2. The microdevice of claim 1, wherein the plurality of first electrodes are arranged in a lattice shape.

3. The microdevice of claim 1, wherein the plurality of first electrodes are mutually electrically connected to each other.

4. The microdevice of claim 1, wherein the first electrode array comprises:
the plurality of electrodes mutually electrically connected to each other; and
a holding pad electrically connected to the plurality of first electrodes and to which a predetermined voltage is applied from the power supply unit.

5. The microdevice of claim 1, wherein the plurality of microwells each have a cylindrical shape or a polygonal column shape.

6. The microdevice of claim 1, wherein the depth and the width of each of the plurality of microwells corresponds to those of a single cell.

7. The microdevice of claim 1, wherein a height of the microchannel of the second electrode corresponds to a diameter of a single cell.

8. A method of fusing cells, the method comprising:
providing the microdevice of claim 1;
injecting first cells through an inlet hole and flowing the first cells through a microchannel;
applying an alternating current (AC) voltage between a first electrode and a second electrode such that the injected first cells are trapped in each microwell according to a dielectrophoresis and gravity;
injecting second cells through the inlet hole and flowing the second cells through the microchannel;
performing electroporation by applying a direct current (DC) voltage between the first electrode and the second electrode when the second cells flow in and are disposed on the microwell in which the first cells are aligned;
applying a quasi-damping AC voltage between the first electrode and the second electrode such that the electroporated first and second cells are fused by being adjacently disposed to each other according to a dielectrophoresis; and
obtaining the fused first and second cells through an outlet hole.
